(19) United States
(12) Patent Application Publication
(10) Pub. No.: US 2017/0368149 A1
(43) Pub. Date: Dec. 28, 2017

(54) METHODS FOR INCREASING SERUM IGF-1 IN AN ANIMAL

(71) Applicant: PURETEIN BIOSCIENCE LLC., St. Lous Park, MN (US)

(72) Inventor: Brett CASEBOLT, Edina, MN (US)

(21) Appl. No.: 15/535,781

(22) PCT Filed: Dec. 16, 2015
(86) PCT No.: PCT/US15/66119

§ 371 Date: Jun. 14, 2017

Related U.S. Application Data
(60) Provisional application No. 62/092,340, filed on Dec. 16, 2014.

(51) Int. Cl. A61K 38/30; A61K 39/395; A23L 33/10

(52) U.S. Cl. CPC A61K 38/30; A23L 33/10; A61K 39/395

(57) ABSTRACT

Provided herein are methods for increasing the concentration of insulin-like growth factor (IGF) in the serum of a subject. In one embodiment, the method includes administering active IGF-1 to a subject, wherein the concentration of IGF in the serum of the subject is increased. Also provided are methods for treating a condition in a subject, methods for improving or maintaining the health of a subject, and methods for improving a characteristic of a subject. Examples of conditions include a motor neuron disorder, Alzheimer's disease, myocardial infarction, hypoxic-ischemic brain injury, osteoporosis, skeletal muscle repair, and growth failure. Examples of characteristics include increased milk production, increased fertilization, increased reproduction, increased growth, increased oocyte quality in a ruminant undergoing superovulation, and increased embryo viability. In one embodiment, the IGF-1 administered to the subject is obtained from a natural source that has been processed to increase the amount of active IGF-1.

### CROSS-REFERENCE TO RELATED APPLICATIONS

This application claims the benefit of U.S. Provisional Application Ser. No. 62/092,340, filed Dec. 16, 2014, which is incorporated by reference herein.

### SUMMARY

Provided herein are methods for increasing the concentration of insulin-like growth factor (IGF) in the serum of a subject. In one embodiment, the method includes administering active IGF-1 to a subject, wherein the concentration of IGF in the serum of the subject is increased compared to the concentration of IGF in the serum the subject before administration of the composition that includes active IGF. The increase may be, for instance, at least 5%, at least 10%, at least 15%, or at least 20%.

In one embodiment, the method is for treating a condition in a subject. The method includes administering active IGF-1 to a subject having or at risk of having a condition. In one embodiment, the condition is a motor neuron disorder, Alzheimer's disease, myocardial infarction, hypoxic-ischemic brain injury, osteoporosis, skeletal muscle repair, or growth failure. In one embodiment, the motor neuron disorder is amyotrophic lateral sclerosis, degeneration of spinal cord motor neurons, or degeneration of invertebral disc cells.

In one embodiment, the method is for improving or maintaining the health of a subject. The method includes administering active IGF-1 to a subject, wherein a health metric of the subject is improved or maintained compared to a control. In one embodiment, the control is the subject before the administering. The health metric of the subject that is improved and/or maintained can be mass and/or fiber size of skeletal muscle, bone growth, brain development, cardiac metabolism and function, immune function, or hair follicle development.

In one embodiment, the method is for improving a characteristic of a subject. The method includes administering active IGF-1 to a subject, wherein a characteristic of the subject is improved compared to the subject before the administration. The characteristic may be increased milk production, increased fertilization, increased reproduction, increased growth, increased oocyte quality in a ruminant undergoing superovulation, or increased embryo viability. In one embodiment, the subject is a ruminant, such as a dairy cow.

In one embodiment, the IGF-1 administered to the subject has been subjected to an activation process that increases the amount of active IGF-1. In one embodiment, the IGF-1 administered to the subject is obtained from a natural source that has been processed to increase the amount of active IGF-1. The natural source may be blood, a blood-derived product, milk, a milk-derived product, colostrum, or a colostrum-derived product. In one embodiment, the natural source may be a combination of two or more of those natural sources or other natural sources.

In one embodiment, the administering include daily administration of at least 0.05 nanograms of active IGF-1 per kilogram bodyweight of the subject daily (ng/kg), at least 0.1 ng/kg, at least 0.5 ng/kg, at least 2 ng/kg, at least 5 ng/kg, at least 10 ng/kg, at least 20 ng/kg, at least 50 ng/kg, or at least 100 ng/kg. In one embodiment, the administering further includes administering inactive IGF-1, wherein at least 20% of the total IGF-1 administered is active IGF-1.

The administering can include feeding the subject a food product that includes the active IGF-1. In one embodiment, the food product is administered to the subject for at least 4 days, for at least 2 weeks, or for at least 1 month. The administering can include parenteral administration, such as intravenous, intradermal, subcutaneous, intraperitoneal, intramuscular administration. The subject may be a bovine species, a porcine species, a cervid species, a canine species, a feline species, a equine species, a ovine species, an avian species, or a human.

### BRIEF DESCRIPTION OF THE FIGURES

FIG. 1. Serum levels of IGF-1 in pigs fed a diet supplemented with active IGF-1.

FIG. 2. Serum levels of IGF-1 in calves fed a diet supplemented with active IGF-1.

### DETAILED DESCRIPTION OF ILLUSTRATIVE EMBODIMENTS

Provided herein are methods for using a composition that includes one or more proteins. In one embodiment, a composition includes insulin-like growth factor (IGF), such as IGF-1 and/or IGF-2. IGF plays a role in regulation of normal physiology and a number of pathological states, including cancer, as well as cell proliferation and inhibition of cell death. IGF may affect different growth stages. Insulin-like growth factor 2 (IGF-2) is thought to be a primary growth factor required for early development while insulin-like growth factor 1 (IGF-1) expression is required for achieving maximal growth. Almost every cell in the human body is affected by IGF-1, including cells in muscle, cartilage, bone, liver, kidney, nerves, skin, and lungs. IGF-1 can also regulate cell growth and development, including in nerve cells, as well as DNA synthesis. IGF proteins are highly conserved between species, and the amino acid sequences of IGF proteins from different species are known and readily available to the skilled person.

Whether a protein is an IGF can be easily determined by the skilled person. For instance, polyclonal and monoclonal antibodies that specifically bind to IGF-1 and/or to IGF-2 are commercially available, and react with IGF from various species including human, equine, canine, bovine, porcine, and avian. These readily available antibodies lack cross-reactivity and/or interference by other closely related proteins and binding proteins. A single antibody or a panel of antibodies that recognizes different regions of an IGF, such as N-terminal, C-terminal, or amino acids present between the ends of the protein, may be used to determine whether a protein is an IGF protein. Methods for determining whether an IGF protein is active are known in the art and routine.

IGF proteins have high sequence similarity to insulin, but unlike insulin, IGF proteins associate with distinct binding proteins present in serum and other biological fluids (Baxter, 2000, Am J Physiol Endocrinol Metab, 278: E967-E976; Hwa et al., 1999, Endocrine Reviews, 20(6):761-787). Most IGF present in products derived from an animal, such as, but not limited to, blood and blood-derived products, milk and milk-derived products, and colostrum and colostrum-derived products, is bound to a binding protein. However, since these binding proteins inhibit the activity of IGF, most IGF present in animal derived products is inactive due to its being bound to a binding protein. For instance, less than 1% of IGF-1 in plasma is not bound to a binding protein (Carel et al., Safety of Recombinant Human Growth Hormone, In: Current Indications for Growth Hormone Therapy, 2nd rev. ed., vol. ed.: Hindmarsh, Karger, Switzerland, p. 48).

An IGF is considered to be active if it is not bound to a binding protein, and is considered to be inactive if it is bound to a binding protein. Active IGF is often referred in the art as free, unbound, bioactive, and/or active. Methods for measuring the concentration of active IGF are known to the skilled person and are routine. Assays are commercially available, including solid phase sandwich ELISA assays that permit measurement of IGF that is not bound to a binding protein (e.g., R&D Systems, Minneapolis, Minn., catalog number DFG100).

A composition useful in the methods described herein includes active IGF, and optionally includes inactive IGF. In one embodiment, a composition is present in a food product. As used herein, a “food product” is a compound or mixture of compounds that can be ingested by a subject, such as a human. A food product may be solid, semi-solid, or liquid. Examples include, but are not limited to, solid and semi-solid dairy products, including fermented dairy products, for instance yogurt. Beverages to which IGF can be added include milk, vegetable juice, fruit juice, soy milk, soybean milk, fermented soybean milk, and fruit flavored dairy beverages. In one embodiment, a food product is a feed for animal use, for instance, for feeding domesticated animals such as companion animals including, but not limited to, dogs or cats, and livestock including, but not limited to, bovine, porcine, avian, cervid, canine, feline, equine, or ovine animals. The appropriate concentration to add to a food product can be determined by the skilled person having knowledge of the level of active IGF in a composition and the approximate amount of food product to be eaten daily by the animal. In those embodiments where the animal is not a human, the skilled person will understand that estimating the amount of feed eaten by an animal is typically based on the average for a population of animals.

In one embodiment, the composition may include a pharmaceutically acceptable carrier. As used herein “pharmaceutically acceptable carrier” includes, but is not limited to, saline, solvents, dispersion media, coatings, antibacterial and antifungal agents, isotonic and absorption delaying agents, and the like, compatible with pharmaceutical administration.

A composition compatible with pharmaceutical administration may be prepared by methods well known in the art of pharmacy. In general, a composition can be formulated to be compatible with its intended route of administration. A formulation may be solid or liquid. Administration may be systemic or local. In some aspects local administration may have advantages for site-specific, targeted disease management. Local administration may provide high, clinically effective concentrations directly to the treatment site, with less likelihood of causing systemic side effects.

Examples of routes of administration include parenteral (e.g., intravenous, intradermal, subcutaneous, intraperitoneal, intramuscular), enteral (e.g., oral), and topical (e.g., epicutaneous, inhalational, transmucosal) administration. Appropriate dosage forms for enteral administration of a composition described herein include tablets, capsules or liquids, as well as a food product. Appropriate dosage forms for parenteral administration may include intravenous administration. Appropriate dosage forms for topical administration may include creams, ointments, and skin patch. Methods for making a pharmaceutically acceptable composition that includes IGF are known to the skilled person (Mahler et al., US Published Patent Application 20110152188).

Compositions can include sterile aqueous solutions or dispersions and sterile powders for the extemporaneous preparation of sterile solutions or dispersions. For intravenous administration, suitable carriers include physiological saline, bacteriostatic water, Cremophor EL™ (BASF, Parsippany, N.J.) or phosphate buffered saline (PBS). A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., the IGF, such as IGF-1) in the required amount in an appropriate solvent with one or a combination of ingredients, as required, followed by filtered sterilization. Generally, dispersions are prepared by incorporating the active compound into a sterile vehicle, which contains a basic dispersion medium and any other appropriate ingredients. In the case of sterile powders for the preparation of sterile injectable solutions, preferred methods of preparation include vacuum drying, spray-drying, and freeze-drying to yield a powder of the active ingredient plus any additional desired ingredient from a previously sterilized solution thereof.

A composition for use in topical administration may be formulated into many types of vehicles. Non-limiting examples of suitable vehicles include emulsions (e.g., oil-in-water, water-in-oil, silicone-in-water, water-in-silicone, water-in-oil-in-water, oil-in-water, oil-in-water-in-oil, oil-in-water-in-silicone, etc.), creams, lotions, solutions (both aqueous and hydro-alcoholic), anhydrous bases (such as lipsticks and powders), gels, ointments, or pastes (Williams, Transdermal and Topical Drug Delivery, Pharmaceutical Press, London, 2003). Variations and other vehicles will be apparent to the skilled artisan and are appropriate for use in the methods described herein.

It is also contemplated that an active compound may be encapsulated for delivery past the rumen of a ruminant or to a target area such as skin. Non-limiting examples of encapsulation techniques include the use of liposomes, vesicles, and/or nanoparticles (e.g., biodegradable and non-biodegradable colloidal particles including polymeric materials in which the ingredient is trapped, encapsulated, and/or absorbed, examples include nanospheres and nanocapsules) that can be used as delivery vehicles to deliver such ingredients to skin or digestive tract.

Oral compositions generally include an inert diluent or an edible carrier. In one embodiment, an oral composition includes a food product. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compound is delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compound is formulated into ointments, salves, gels, or creams as generally known in the art.

The active compound can also be prepared in the form of suppositories (e.g., with conventional suppository bases such as cocoa butter and other glycerides) or retention enemas for rectal delivery.

Pharmaceutical administration can be one or more times per day to one or more times per week, including once every other day. The skilled artisan will appreciate that certain factors may influence the dosage and timing required to effectively treat a subject, including, but not limited to, the severity of the infection, previous treatments, the general health and/or age of the subject, and other diseases present.

IGF useful in the methods described herein is obtainable from various sources. In one embodiment, a source is a natural source, such as a biological material from an animal. Examples of animals include, but are not limited to, vertebrates. Examples of vertebrates include, but are not limited to, mammals, such as a species that is bovine, porcine, cervid, canine, feline, equine, ovine, or a human. Another example of a vertebrate is an avian species. Examples of biological materials include, but are not limited to, blood and blood-derived products (e.g., whole blood, red blood cells, plasma, and derivatives thereof); milk and milk products (e.g., liquid milk, powdered milk, cheese, whey and whey products, curd, cheese, casein, lactose, milk fat, and derivatives thereof); colostrum and colostrum-derived products (e.g., liquid colostrum, dried colostrum); egg and egg-derived products (e.g., egg yolk, egg whites, egg membranes), bodily fluids (e.g., saliva, semen), and tissues (e.g., mucosa tissue, intestinal tissue, embryonic tissue). Examples of plasma include, but are not limited to, dried plasma and liquid plasma and fractions thereof, such as a lipid fraction. Examples of whey products include, but are not limited to, liquid whey, whey protein concentrate, whey protein isolate, whey cream, whey retentate, procream, deproteinized whey, and delactosed permeate. Examples of colostrum-derived products include, but are not limited to, liquid colostrum whey, colostrum whey protein concentrate, colostrum whey protein, colostrum whey cream, colostrum whey retentate, colostrum procream, colostrum deproteinized whey, colostrum delactosed permeate, colostrum casein, colostrum lactose, and colostrum curd. In one embodiment, the colostrum is colostrum secreted by a female within the first 6, the first 12, the first 24, or the first 48 hours after birth of offspring. In one embodiment, a natural source of IGF useful in the methods described herein is not colostrum. In one embodiment, IGF useful in the methods described herein is produced using recombinant techniques, or chemically or enzymatically synthesized. As used herein, IGF from a natural source, for instance, blood or a blood-derived product, is not produced using recombinant techniques, or chemically or enzymatically synthesized. Biological material, such as blood or a blood-derived product, useful for producing a composition with active IGF is readily available commercially.

A biological material may be enriched for the amount of total IGF present. A protein is enriched if it is present in a significantly higher fraction compared to the biological material from which the protein was enriched. The higher fraction may be, for instance, an increase of 2-fold, 4-fold, 6-fold, 10-fold, 100-fold, 1,000-fold, or 10,000-fold. Enrichment may result from reducing the amount of other molecules present in the biological material, e.g., proteins. However, the term enriched does not imply that there are no other molecules, e.g., proteins, present. Enriched simply means the relative amount of IGF has been significantly increased. The term “significant” indicates that the level of increase is useful to the person making such an increase. Enrichment of IGF is the result of intervention by a person to elevate the proportion of the protein.

Optionally, IGF can be purified from a biological material. A protein is considered to be purified if at least 75%, least 85%, or at least 95% of other components present in the biological material are removed. Proteins that are produced through chemical or recombinant means are considered to be purified. Methods for enriching and/or purifying IGF are known to the skilled person and are routine. Non-limiting examples of such procedures include fractionation on immunoaffinity or ion-exchange columns, ethanol precipitation, reverse phase HPLC, chromatography on silica or on an ion-exchange resin such as DEAE, chromatofocusing, SDS-PAGE, ammonium sulfate precipitation, ligand affinity chromatography, and gel filtration using, for example, cross-linked gels and/or hollow fiber.

Most, e.g., 95% to 99%, of the IGF obtained from many natural sources is associated with binding protein that causes the IGF to be inactive. Optionally, the amount of active IGF in a composition that is obtained from a natural source can be increased, e.g., the amount of total IGF in the composition may be substantially unchanged but the amount of active IGF is increased, such that the amount of active IGF as a percentage of the total IGF is increased. Methods for increasing the amount of IGF that is active include processes routinely used to activate functional proteins obtained from a biological material. Such activation processes include, but are not limited to, exposing the biological material to heat shock, temperature adjustment, alcohol extraction, pH adjustment, enzyme addition, ionic changes, other chemical additions, and pressure, or combinations thereof (Daughaday et al., 1989, Endocr Rev. 10:68-91; Daughaday et al., 1987, J Lab Clin Med. 109:355-363; Breier et al., 1991, J Endocrinol. 128:347-357). Without intending to be limited by theory, such methods typically cause the dissociation of the binding protein from the IGF protein.

In one embodiment, the amount of active IGF in a composition that is obtained from a natural source can be increased by at least 2-fold, at least 4-fold, at least 5-fold, at least 10-fold, at least 20-fold, at least 50-fold, or at least 100-fold compared to the amount of active IGF in the composition before it is processed to activate IGF. In one embodiment, at least 20%, at least 30%, at least 40%, at least 50%, at least 60%, at least 70%, at least 80%, at least 90%, or 100% of the total IGF present is active. In one embodiment, no greater than 80%, no greater than 70%, no greater than 60%, no greater than 50%, no greater than 40%, no greater than 30%, no greater than 20%, no greater than 10%, or no greater than 5% of the total IGF present is inactive (e.g., bound to a binding protein). The composition subjected to the processing can be, for instance, a biological material from an animal, such as a blood or blood-derived product. Optionally, the biological material may be one that has been enriched for total IGF. Products made from natural sources and processed to activate IGF are commercially available as the product BETAGRO and IMMUTEIN (GBH Labs, Maple Grove, Minn.).

In those embodiments where the source of IGF is a natural source, the composition may typically include other components, including other proteins. Examples of other proteins that may be present include, but are not limited to, lysozymes, lactoferrin, growth factors, transfer factors, cytokines, and immunoglobulins.

Also provided herein are methods for using a composition described herein. In one embodiment, a method includes administering a composition described herein to an animal. Examples of animals include, but are not limited to, vertebrates. Examples of vertebrates include, but are not limited to, mammals, such as an animal that is bovine (such as a domesticated cow), porcine (such as a domesticated pig), cervid (such as a deer), canine (such as a domesticated dog), feline (such as a domesticated cat), equine (such as a domesticated horse), ovine, or human. In one embodiment, the animal is one used as a source of dairy products, such as a dairy cow or a goat. Another example of a vertebrate is an avian species (such as domesticated fowl). The animal may be at an age that is between birth and weaning, between post-weaning and adulthood, or a mature (adult) animal. The animal may be a female or a male.

In one embodiment, the method includes increasing the concentration of total IGF (both active and inactive IGF) in the serum of a subject. The method includes administering to the subject an effective amount of a composition that includes active IGF. The IGF may be IGF-1, IGF-2, or a combination thereof, and the IGF that is increased in the serum may be IGF-1, IGF-2, or a combination thereof.

In one embodiment, the concentration of IGF in the serum of a subject is increased in comparison to a control. A control is an animal that is comparable to the subject except for being administered the composition that includes active IGF. In one embodiment, the concentration of IGF in the serum of a subject is increased in comparison to the subject before the administration. In one embodiment, the increase is a statistically significant increase. In one embodiment, the concentration of IGF in the serum of a subject is increased by at least 5%, at least 10%, at least 15%, at least 20%, at least 25%, at least 30%, at least 35%, at least 40%, at least 45%, at least 50%, at least 55%, at least 75%, at least 100%, or at least 125% compared to the concentration of IGF in the serum a subject that was not administered the composition that includes active IGF. In one embodiment, the concentration of IGF in the serum of a subject is increased by at least 5%, at least 10%, at least 15%, at least 20%, at least 25%, at least 30%, at least 35%, at least 40%, at least 45%, at least 50%, at least 55%, at least 75%, at least 100%, or at least 125% compared to the concentration of IGF in the serum of the subject before administration of the composition that includes active IGF. In one embodiment, the amount of IGF in the serum of a subject is increased by no greater than 300%, no greater than 250%, no greater than 200%, no greater than 175%, or no greater than 150% compared to the concentration of IGF in a subject that was not administered the composition that includes IGF.

In one embodiment, the method is for treating a condition in an animal. The method includes administering an effective amount of a composition to a subject having or at risk of having a condition, or exhibiting symptoms and/or clinical signs of a condition. Optionally, the method includes determining whether a symptom and/or clinical sign of the condition is changed. In one embodiment, a symptom and/or clinical sign is reduced. Examples of conditions that can be treated by increased serum levels of IGF are known to the skilled person. Examples of conditions include, but are not limited to, motor neuron disorders including, for instance, amyotrophic lateral sclerosis and degeneration of spinal cord motor neurons and invertebral disc cells (Lewis et al., 1993, Exp. Neurol., 124(1):73-88; Gruber et al., 2000, Spine, 25(17):2153-2157; Kaspar et al., 2003, Science, 301(5634):839-842), Alzheimer's disease (Gasparini et al., 2003, Trends in Neurosci., 26(8):404-406), myocardial infarction (David et al., 2006, PNAS USA, 103(21):8155-8160), hypoxic-ischemic brain injury (Guan et al., 1993, J Cerebral Blood Flow and Metab., 13:609-616; Guan et al., 1996, Brain Devel., 137(3):893-898), osteoporosis (Yakar et al., 2002, J. Clin, Invest., 110(6):771-781; Ljunghall etl al., 1992, J. Internal Med., 232:59-64), skeletal muscle repair (Song et al., 2013, Trends Endocrinol. Metab., 24(6):310-319), and growth failure (Collett-Solberg et al., 2008, J. Clin. Endocrinol. Metab., 93:10-18).

Treatment of a condition can be prophylactic or, alternatively, can be initiated after the development of a condition. As used herein, the term “symptom” refers to subjective evidence of a condition experienced by the subject. As used herein, the term “clinical sign” or, simply, “sign” refers to objective evidence of a condition. Symptoms and/or clinical signs associated with a condition that can be treated by increased serum levels of IGF and the evaluations of such symptoms vary depending upon the condition, and are routine and known in the art.

Treatment that is prophylactic, for instance, initiated before a subject manifests symptoms or signs of a condition that can be treated by increased serum levels of IGF, is referred to herein as treatment of a subject that is “at risk” of developing the condition. Accordingly, administration of a composition can be performed before, during, or after the occurrence of the condition. Treatment initiated after the occurrence of a condition may result in decreasing symptoms of the condition, and/or completely removing symptoms of the condition.

In one embodiment, the method is for improving and/or maintaining the health of a subject. In one embodiment, improvement and/or maintenance of health is in comparison to a control subject. In one embodiment, the improvement and/or maintenance of health of a subject is in comparison to the subject before the administration. The method includes administering an effective amount of a composition to a subject. Examples of a subject's health that can be improved and/or maintained by increased serum levels of IGF are known to the skilled person. Examples include, but are not limited to, mass and fiber size in skeletal muscle (Stitt et al., 2004, Mol. Cell, 14:395-403), bone growth (Isgaard et al., 1986, Am. J. Physiol. Endocrinol. Metab., 25:E367-E372; Wang et al., 1999, FASEB J., 13:1985-1990), brain development (Cheng et al., 2000, PNAS USA, 97:10236-10241), cardiac metabolism and function (Lausten et al., 2007, Mol. Cellul. Biol., 27:1649-1664), immune function (Ni et al., 2013, Nat. Comm., 4:1479), and hair follicle development (Le et al., 2014, Growth Hormone and IGF Res., 2-3:89-94). In one embodiment, the health aspect is weight gain, such as average daily gain, average daily feed intake, and/or feed conversion, for an agricultural animal.

Improving and/or maintaining the health of a subject can be prophylactic or, alternatively, can be initiated after an aspect of the subject's health has declined. Indicators of the health of a subject that can be aided by increased serum levels of IGF and the evaluations of such indications are routine and known in the art.

In one embodiment, the method is for improving certain characteristics of a subject. In one embodiment, improvement of a characteristic is in comparison to a control subject. In one embodiment, the improvement of a characteristic of a subject is in comparison to the subject before the administration. The method includes administering an effective amount of a composition to an animal. In one embodiment, the animal is a ruminant, such as a bovine (e.g., a cow or calf) or a cervid (e.g., a deer or elk). Examples of certain characteristics of an animal that can be improved by increased serum levels of IGF are known to the skilled person. Examples include, but are not limited to, increased milk production, such as in dairy cows (Prosser et al., 1989, J. Darty Res., 56(1):17-26), increased fertilization, reproduction, and/or growth (McGuire, 1992, J. Amim. Sci., 70:2901-2910), and increased oocyte quality and embryo viability, such as in domestic ruminants undergoing superovulation (Velazquez et al., 2009, Reproduction, 137:161-180).

In one embodiment, the administering can be feeding a composition that includes active IGF to the animal. In one embodiment, active IGF can be present in a food product. The food product may naturally include the active IGF, or the food product may be supplemented with active IGF. In one embodiment, the addition of active IGF occurs by the supplementation of a food product with a biological material, such as a blood-derived product, e.g., plasma, that has been processed to increase the amount of active IGF. The amount of active IGF administered by feeding on a daily basis may be at least 0.05 ng/kg, at least 0.1 ng/kg, at least 0.5 ng/kg, at least 2 ng/kg, at least 5 ng/kg, at least 10 ng/kg, at least 20 ng/kg, at least 50 ng/kg, or at least 100 ng/kg, where ng refers to nanograms of active IGF and kg refers to kilograms bodyweight of the animal. In one embodiment, the amount of active IGF administered by feeding on a daily basis is no greater than 150,000 ng/kg, no greater than 100,000 ng/kg, no greater than 50,000 ng/kg, or no greater than 20,000 ng/kg, where ng refers to nanograms of active IGF and kg refers to kilograms bodyweight of the animal. The active IGF administered may be active IGF-1, active IGF-2, or a combination thereof. In one embodiment, the active IGF administered is active IGF-1. In one embodiment there is no upper limit on the amount of active IGF administered.

In one embodiment, the feed is provided to an animal for at least 1 day, at least 4 days, at least 7 days, at least 2 weeks, at least 3 weeks, at least 1 month, at least 2 months, or at least 3 months. In one embodiment, the feed may be provided to an animal for no greater than 3 months, no greater than 2 months, no greater than 1 month, no greater than 3 weeks, or no greater than 2 weeks. Thus, in one embodiment, the period of time may be at least 1 day and no greater than 3 months, or any combination of time periods selected between those numbers. In one embodiment, the feed may be provided to an animal as part of its diet throughout its life.

In one embodiment, the administering can be parenteral or topical. The amount of active IGF to be administered by a parenteral or topical route in the methods described herein can be determined by standard pharmaceutical procedures in cell cultures or experimental animals, e.g., for determining the ED₅₀ (the dose therapeutically effective in 50% of the population). The data obtained from cell culture assays and animal studies can be used in formulating a range of dosage for use in an animal. The dosage of active IGF lies preferably within a range that includes the ED₅₀ with little or no toxicity; however, it is expected that high levels of active IGF will not be detrimental to an animal. The dosage may vary within this range depending upon the dosage form employed and the route of administration utilized. For a compound used in the methods of the invention, the therapeutically effective dose can be estimated initially from cell culture assays and/or experimental animals. A dose may be formulated in animal models to achieve a circulating plasma concentration of active IGF that is increased by at least 5%, at least 10%, at least 15%, at least 20%, at least 25%, at least 50%, at least 75%, at least 100%, at least 125%, or at least 150%.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### Example 1

The following trial was conducted to determine if feeding a composition that includes active IGF-1 caused a change in the level of IGF-1 in plasma.

At weaning 14 pigs were divided equally into two groups, a control group and a test group. Immediately after weaning all pigs were fed a standard diet for 25 days. The standard diet of the control group was supplemented with spray dried animal plasma. The standard diet of the test group was supplemented with betaGRO, a composition that contained active IGF-1, at a concentration that resulted in daily ingestion of approximately 3-32 nanograms of active IGF-1 per kilogram bodyweight (ng/kg) of an animal. The composition was added at 0.1% to 0.3% (approximately 1 pound per ton to 7 pounds per ton).

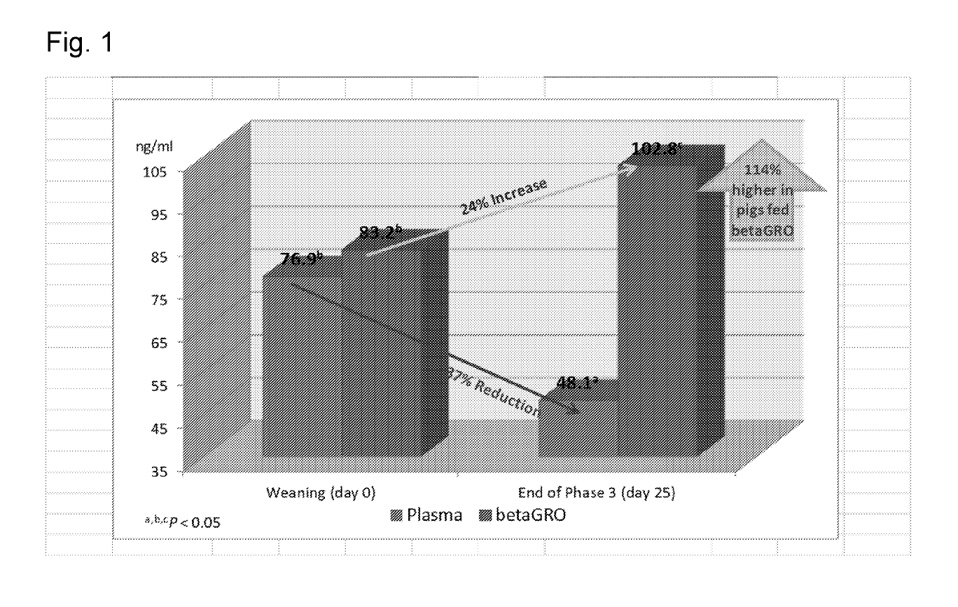

Serum levels of IGF-1 in all pigs was determined at the beginning of the trial and 25 days later at the end of the trial. The method used to determine IGF-1 was a commercially available ELISA. As shown in FIG. 1, the levels of serum IGF-1 in the two groups of pigs was similar at weaning, but by the end of the trial the level of IGF-1 in the pigs in the test group was 114% higher than the IGF-1 level in pigs of the control group (p-value <0.05). Further, the average daily gain (in pounds) for pigs in the test group was 0.7 pounds, and the average daily gain for pigs in the control group was 0.47 pounds.

The results indicate that the level of IGF-1 in the serum of an animal can be significantly increased by feeding a composition that includes active IGF-1, and that the average daily gain of pigs can be increased when fed a composition that includes IGF-1.

### Example 2

To determine if the concentration of serum IGF-1 could be increased by feeding calves a composition that includes active IGF-1, betaGRO, a composition that contained active IGF-1, was mixed with milk replacer just prior to each feeding. The two calves were fed at about 0400 and 1600 each day, and were fed about 18 mg/kg of betaGRO at each of the two daily feedings. Each calf ingested an average of 10.1 nanograms of active IGF-1 per kilogram bodyweight per day. There was no indication the product had any negative impact on consumption as calves readily consumed the mixed product. Jugular blood samples were collected daily between 0800 and 1000. Serum IGF-I was determined with a validated bovine radioimmunoassay.

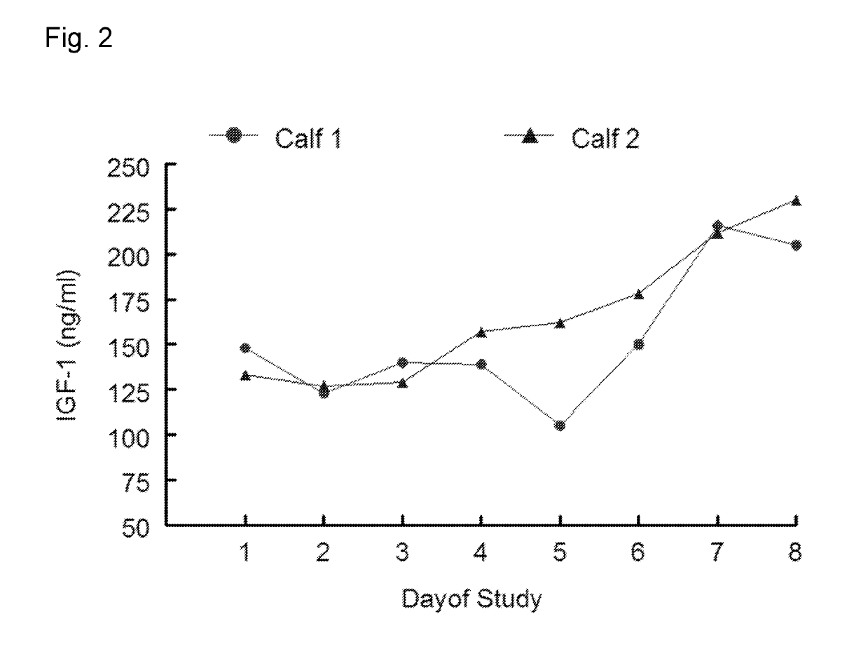

As shown in FIG. 2, the concentration of IGF-1 in serum increased in each calf, and the increase was evident as early as day 4 in one calf.

1. A method for increasing the concentration of IGF in the serum of a subject, the method comprising administering active IGF-1 to a subject, wherein the concentration of IGF in the serum of the subject is increased compared to a control subject.

2. The method of claim 1 wherein the IGF-1 administered to the subject has been subjected to an activation process that increases the amount of active IGF-1.

3. The method of claim 1 wherein the IGF-1 administered to the subject is obtained from a natural source that has been processed to increase the amount of active IGF-1.

4. The method of claim 1 wherein the administering further comprises administering inactive IGF-1, wherein at least 20% of the total IGF-1 administered is active IGF-1.

5. The method of claim 3 wherein the natural source is blood or a blood-derived product.

6. The method of claim 3 wherein the natural source is milk or a milk-derived product.

7. The method of claim 3 wherein the natural source is colostrum or a colostrum-derived product.

8. The method of claim 1 wherein the administering comprises daily administration of at least 0.05 nanograms of active IGF-1 per kilogram bodyweight of the subject daily (ng/kg), at least 0.1 ng/kg, at least 0.5 ng/kg, at least 2 ng/kg, at least 5 ng/kg, at least 10 ng/kg, at least 20 ng/kg, at least 50 ng/kg, or at least 100 ng/kg.

9. The method of claim 1 wherein the concentration of IGF in the serum of the subject is increased by at least 5%, at least 10%, at least 15%, or at least 20% compared to the subject before the administration.

10. The method of claim 1 wherein the administering comprises feeding the subject a food product comprising the active IGF-1.

11. The method of claim 10 wherein the food product is administered for at least 4 days, for at least 2 weeks, or for at least 1 month.

12. The method of claim 1 wherein the subject is a bovine species, a porcine species, a cervid species, a canine species, a feline species, a equine species, a ovine species, an avian species, or a human.

13. The method of claim 1 wherein the subject is a porcine species that is an age between post-weaning and adulthood, or is an adult.

14. The method of claim 1 wherein the administering comprises parenteral administration.

15. The method of claim 14 wherein the parenteral administration is selected from intravenous, intradermal, subcutaneous, intraperitoneal, and intramuscular.

16. A method for treating a condition in a subject, the method comprising:
administering active IGF-1 to a subject having or at risk of having a condition.

17. The method of claim 16 wherein the condition is selected from a motor neuron disorder, Alzheimer's disease, myocardial infarction, hypoxic-ischemic brain injury, osteoporosis, skeletal muscle repair, and growth failure.

18. The method of claim 17 wherein the motor neuron disorder is selected from amyotrophic lateral sclerosis, degeneration of spinal cord motor neurons, and degeneration of invertebral disc cells.

19.-32. (canceled)

33. A method for improving or maintaining the health of a subject, the method comprising:
administering active IGF-1 to a subject, wherein a health metric of the subject is improved or maintained compared to the subject before the administration.

34. The method of claim 33 wherein a health metric of the subject that is improved and/or maintained is selected from mass and/or fiber size of skeletal muscle, bone growth, brain development, cardiac metabolism and function, immune function, and hair follicle development.

35.-48. (canceled)

49. A method for improving a characteristic of a subject, the method comprising:
administering active IGF-1 to a subject, wherein a characteristic of the subject is improved, wherein the characteristic is selected from increased milk production, increased fertilization, increased reproduction, increased growth, increased oocyte quality in a ruminant undergoing superovulation, and increased embryo viability.

50.-63. (canceled)

64. The method of claim 49 wherein the subject is a ruminant.

65. The method of claim 64 wherein the ruminant is a dairy cow.
